SEQUENCE LISTING (5 sequences)

SEQ ID NO: 1 - DNA, 28 bp, Artificial Sequence
  Description of Artificial Sequence Oligonucleotide CF10
gtatctatattcatcataggaaacacca

SEQ ID NO: 2 - DNA, 27 bp, Artificial Sequence
  Description of Artificial Sequence Oligonucleotide CF1629.27
aagatgatattttctttaatggtgcca

SEQ ID NO: 3 - DNA, 24 bp, Artificial Sequence
  Description of Artificial Sequence Oligonucleotide 508CF
atgatattttctttaatggtgcca

SEQ ID NO: 4 - DNA, 56 bp, Artificial Sequence
  Description of Artificial Sequence Oligonucleotide MB65.56
cacgggctagcgctttcgcgctctcccaggtgacgcctcgtgaagaggcgcgacct

SEQ ID NO: 5 - DNA, 61 bp, Artificial Sequence
  Description of Artificial Sequence Oligonucleotide capture probe
ggggaccccccggaaggggggacgaggtgcgggcacctcgtacgggagttcgaccgtgaca

(19) United States
(12) Patent Application Publication
(10) Pub. No.: US 2002/0098485 A1
(43) Pub. Date: Jul. 25, 2002

(54) Method for nucleic acid detection via Qbeta replicase

(76) Inventors: Ann M. Morello, Cambridge, MA (US); Qingping Jiang, Northborough, MA (US); John E. Monahan, Walpole, MA (US); Say-Jong Law, Westwood, MA (US)

Correspondence Address: REED & ASSOCIATES, 800 MENLO AVENUE, SUITE 210, MENLO PARK, CA, 94025, US

(21) Appl. No.: 09/781,106

(22) Filed: Feb. 8, 2001

Related U.S. Application Data
(60) Provisional application No. 60/180,918, filed on Feb. 8, 2000.

(51) Int. Cl. C12Q 1/68; C12P 19/34

(52) U.S. Cl. 435/6; 435/91.2

(57) ABSTRACT

Disclosed is amplification of a target sequence using the activity of a replicase that quasi-autocatalytically replicates a specific replicase replicable or “replicatable” sequence, while ensuring fidelity thereof by detecting the presence of the amplified target or antitarget sequence rather than the amplified replicase replicable sequence. A method according to the invention for assaying a target nucleic acid comprising hybridizing a set of one or more amplification probes with a nucleic acid sample is provided. A set of two chemiluminescent detection probes each for detecting portions of amplified target sequence along with a dual photomultiplier tube detection system for simultaneous detection of the two chemiluminescent detection probes are also provided for practicing the invention, for use with a target specific set of amplification probes. Kits for practicing the invention are also provided.

### CROSS-REFERENCE TO RELATED APPLICATIONS

This application claims priority to U.S. Provisional Patent Application Serial No. 60/180,918, filed Feb. 8, 2000.

### FIELD OF THE INVENTION

The present invention is directed to a method and kit for amplifying nucleic acid analytes for detection in a test sample with high specificity. More specifically, the present invention relates to detecting nucleic acid analytes using RNA replicases, and ensuring the fidelity of the amplification using a labeled probe molecule. Typically luminescent probes are employed and a simultaneous detection of dually employed chemiluminescent probes employing dual photomultiplier tubes is employed therewith.

### BACKGROUND OF THE INVENTION

The ability to detect specific target nucleic acid analytes using nucleic acid probe hybridization and nucleic acid amplification methods has many applications. These applications include: diagnoses of infectious or genetic diseases or cancers in humans or other animals; identification of viral or microbial contamination in cosmetics, foods, pharmaceuticals or water; and identification or characterization of, or genetic discrimination between individuals, for diagnosis of disease and genetic predisposition to disease, forensic or paternity testing and genetic analyses for breeding or engineering stock improvements in plants and animals.

The basis of nucleic acid probe hybridization methods and applications is specific hybridization of an oligonucleotide or a nucleic acid fragment probe to form a stable, double-stranded hybrid through complementary base-pairing to particular nucleic acid sequence segments. Particular nucleic acid sequences may occur only in a species, strain, individual organism, or cells taken from an organism.

The basic limitations of nucleic acid probe hybridization assays have been the sensitivity and fidelity of the assays. These depend on the ability of a probe to selectively bind a target molecule, and the magnitude of signal(s) detectable in the time period of detection generated by each probe binding a target molecule relative to the background noise. Known detection methods in the assay art include methods dependent on signals generated by a probe, as by fluorescent moieties or radioactive isotopes comprising the probe. Alternatively, an enzyme, such as alkaline phosphatase or peroxidase, linked to the probe, after probe hybridization and separation of unhybridized probes from hybridized probes, is incubated with specific substrate to produce a characteristic and easily identifiable product. However, the practical detection limit of these assays is about 200,000 target molecules, a detection limit insufficiently sensitive for many applications. Much effort is therefore devoted to increasing the sensitivity of detection systems for nucleic acid probe hybridization assays and increasing the fidelity or specificity of such assays.

A powerful amplification/detection procedures for nucleic acids entails indirect amplification of an amplification probe comprising an antitarget sequence and a replicase replicable sequence rather than direct amplification of a segment or segments of target nucleic acid analytes using the RNA-dependent RNA polymerase activity of the Qβ replicase enzymes. Reference is made to T. Blumenthal and G. G. Carmichael (1979) Ann. Rev. Biochem. 48:525-548; PCT Patent Publication No. WO 87/06270 and U.S. Pat. No. 4,957,858 to B. Chu et al.; G. Feix and H. Sano (1976) Febs. Letters 63:201-204; F. R. Kramer and P. M. Lizardi (1989) Nature 339:401-402; U.S. Pat. No. 4,786,600 to Kramer; P. M. Lizardi et al. (1980) Biotechnology 6:1197-1202; and W. Schaffner et al. (1977) J. Mol. Biol. 117:877-907, for a further description of this procedure. In the procedure, a replicase enzyme replication competent or replicable (sometimes referred to as “replicatable”) nucleic acid sequence is covalently joined or linked to a specific hybridizing probe, e.g., a single-stranded nucleic acid with a sequence complementary to of a target nucleic acid analyte sequence (“target sequence”) being probed for in a sample, termed an anti-target sequence segment. The assembly, comprising an amplification, probe, may be an anti-target sequence segment embedded within a recombinant replicase replicable RNA sequence, or attached to one of the ends of a replicatable sequence. The probe-replicable RNA complex or amplification probe hybridizes by a sequence complementary to a target nucleic acid analyte in the sample.

Hybridized probe-RNA complexes are then typically separated from unhybridized probes. Those replicase replicable sequences of the hybridized probe-target RNA complexes are amplified exponentially by incubation with Qβ replicase (typically after separation from the unhybridized amplification probe sequences). Qβ replicase quasi-autocatalyzes replication of the replicatable nucleic acid sequence (“replicase replicable sequence” competent to form a complex substrate for replication by an RNA replicase) to produce up to 10⁹ reporter molecules (comprising replicatable RNAs) for each hybridized target molecule. Such amplification requires 30 minutes at room temperature, without requiring expensive and inconvenient thermocycling as do other nucleic acid amplification methods, notably the polymerase chain reaction (PCR).

Quasi-autocatalytic replicases such as Qβ replicase are template-specific DNA or RNA directed RNA polymerases. The normal function of Qβ replicase in vivo is to replicate the RNA genome of the Qβ replicase bacteriophage to produce progeny. Each infectious Qβ virion contains one molecule of single stranded RNA of molecular weight 1.5×10⁶, which is defined as the viral plus (+) strand. This is the strand utilized as mRNA to direct viral protein synthesis. Using the +strand as a template, Qβ replicase produces a complementary RNA molecule copy to the +template strand termed a minus (-) strand. Significantly, both + and −strands are templates for the Qβ replicase enzyme. Consequently replication of the RNA template proceeds geometrically as the number of templates doubles with each replication round.

Qβ and other replicases are also known to be capable of utilizing DNA as a template, or a template comprising both deoxyribo- and ribo- nucleotides (“D/RNA”). The use of DNA templates is extremely useful in diagnostic settings involving Qβ replicase, because DNA templates are much less expensive than RNA templates, and are less susceptible to degradation. Other templates for replicases that may be employed include synthetic nucleic acid sequences, for example protein nucleic acids (PNAs) and the like.

The specificity of Qβ replicase for RNAs having certain structural and sequence requirements for quasi-autocatalytic replication ensures that usually only hybridizing amplification probe(s) comprising the complete replicatable RNA sequence (replicase replicable RNA sequence) is amplified (Kramer and Lizardi (1989) supra). Various problems have arisen in the use of quasi-autocatalytic replicases such as Qβ replicase to amplify hybridizing target by way of anti-target comprising the amplification probe along with replicase replicable sequence. Such problems primarily arise because the replicase is a prolific replicator that sometimes can replicate unhybridized copies of nucleic acid sequence. Sequences comprising the replicase replicable sequence and a putative anti-target sequence that does not hybridize because the complementary target sequence that is absent from the sample may be replicated by the Qβ replicase. The probability of some non-hybridizing or partially hybridizing putative anti-target sequences being replicated and amplified as anti-target/target creates a level of background noise that precludes discerning whether a given target sequence being probed for is actually present in the analyte.

Assay sensitivity is a function not only of the amount of signal generated for a given amount of target nucleic acid, but also of the amount of background noise generated in the absence of target nucleic acid. One of the significant sources of noise in nucleic acid amplification systems employing replicases such as the Qβ replicase is replication of unhybridized copies of nucleic acid sequences present in the assay system by the Qβ replicase.

Many systems have been devised to overcome this background signal problem. Some have relied on splitting the complete replicase replicable sequence into two amplification probes, so that the two amplification probes must both hybridize the target segment adjacent to each other for replication. This decreases the probability of replication of an unhybridized or single amplification probe hybridized nucleic acid complex that does not comprise the complex template required for replication by a replicase. However, due to the prolific replication catalyzed by the Qβ replicase, background signal remains a problem even when multiple amplification probes are required to form the complete complex, hybridized template required for replication because of possible amplification, for example, of incomplete complex templates and/or of the presence in the analyte sample of sequence sufficiently similar to the probed for target sequence to form an adequate complex template for amplification.

There remains a need for a method to harness the economical, efficient and specific ability of a replicase enzyme to amplify a desired nucleic acid segment while maintaining the fidelity of that amplification.

### SUMMARY OF THE INVENTION

The present invention allows amplification of a target nucleic acid sequence by employing a quasi-autocatalytic replicase activity, while ensuring fidelity of amplification by use of a method for detecting the presence of the amplified target rather than the amplified replicase replicable sequence.

Therefore an object of the invention is to provide a method for assaying a target nucleic acid comprising combining one or more amplification probes with a nucleic acid sample under conditions suitable for hybridization such that the amplification probe, or probes together if more than one probe is used, hybridize to the target sequence. If more than one amplification probe is employed, each probe comprises an anti-target sequence segment, such that each amplification probe hybridizes to a portion of the target sequence of interest that is being probed for in the nucleic acid sample, with each amplification probe comprising an anti-target sequence segment and a replicase replicable sequence segment.

When more than one amplification probe is employed, the replicase replicable sequence segments of the amplification probes taken together, also have the sequence of, or a complementary sequence to, an RNA sequence which is quasi-autocatalytically replicable by an RNA replicase, resulting in quasi-autocatalytic replication of the entire sequence of the target sequence segments along with the replicase replicable sequence segments of the amplification probes. The anti-target segments are then detected using one or more detection probe molecules, preferably a detection probe comprises a luminescent probe, most preferably a chemiluminescence probe. One or more detection probes may be provided for the amplified anti-target/target sequence segment of each amplification probe employed.

Additional detection probes are provided by the invention for determining the amount of unhybridized replicase replicable sequence such that the signal to noise ratio (SIN) between the amplified target segments (signal) and amplified unhybridized probe sequence (noise) can be determined to measure amplification fidelity.

Another object of the invention is to provide a multiple amplification probe assay system in combination with multiple anti-target/target sequence detection probes. Preferably, the number of detection probes corresponds to the number of amplification probes employed, and detection of anti-target/target sequence by all probes is by simultaneous hybridization of detection probes with amplified assay products, and simultaneous detection. Another object of the invention is to provide multiple detection probes for simultaneous hybridization and detection wherein simultaneous hybridization of the detected sequence with the multiple detection probes effects, for a given detection probe in the presence of an additional detection probe, an increase in the slope of the S/N obtained from use the given probe versus the amount of its corresponding target sequence segment to be detected compared to the slope of signal to noise ratio obtained for the identical detecting absent the additional detection probe.

Yet another object of the invention is to provide kits for performing the methods of the invention.

### BRIEF DESCRIPTION OF THE DRAWINGS

FIG. 1 shows the emission spectrum of LEAE-Bz, an analog of LEAE-NHS.

FIG. 2 shows the emission spectrum of DMAE-Bz, which is an analog of DMAE-CO₂H, the compound used in the synthesis of 5′-DMAE-B65.56

FIG. 3 shows the transmittance spectrum of the Corion custom filter fitted on the PMT employed to detect the DMAE emission signal.

FIG. 4 shows the transmittance spectrum of the Corion filter fitted on the PMT employed to detect the LEAE emission signal.

FIG. 5 is a graph illustrating the signal to noise ratio of the detection for various probes vs. the percent of medivariant cystic fibrosis (MDV-CF) sequence.

FIG. 6 is an image of a gel electrophoresis of Qβ replicase amplified product.

FIG. 7 is a graph illustrating signal to noise ratio vs. the amount of Qβ replicase-amplified MDV-CF.

### DETAILED DESCRIPTION OF THE INVENTION

The following terms are appropriate for definition herein.

The term “amplifiable segment” as used in this application refers to a replicase replicable nucleic acid sequence. Such sequences are competent for replication and consequently amplification without priming and are thus “auto-initiating” and may be termed “autocatalytic” or “quasi-autocatalytic.” The term “autocatalytic” has a meaning synonymous with the term “quasi-autocatalytic” the term is intended to have the same meaning as in literature describing replication by replicase enzymes as exemplified by the Qβ replicase. The term “quasi-autocatalytic” (and “(quasi-)autocatalytic” denoting “autocatalytic or more correctly quasi-autocatalytic”) is employed to distinguish the process from truly autocatalytic processes as when a ribozyme replicates itself.

The phrase “anti-target sequence” as used in this application refers to a complementary nucleic acid sequence to a target site, the target site being a nucleic acid sequence that is to be detected.

The term “luminescent molecule” as used in this application denotes any molecule capable of emitting light. The light emitted is typically at a characteristic frequency and corresponding wavelength of light that is sensitive to the chemical environment and bonds, ligand interaction or complexation of the light emitting moiety. Examples of luminescent molecules include without limitation, chemiluminescent molecules, fluorescent molecules, both having a characteristic absorption frequency which is also environmentally sensitive and phosphorescent molecules.

The term “chemiluminescent molecule” as used in this application denotes a luminescent molecule, e.g. one capable of emitting light, that emits a characteristic frequency and corresponding wavelength of light as a result of the generation of electronically excited states formed as a result of a chemical reaction and therefore in response to a chemical agent capable of generating the electronically excited states by so reacting. Examples of chemiluminescent molecules include without limitation, acridinium compounds, benzacridinium compounds, quinolinium compounds, isoquinolinium compounds, phenanthridinium compounds, luminol compounds, isoluminol compounds, and lucigenin compounds.

The phrase “conditions suitable for catalysis” or “conditions effective for catalysis” or “conditions suitable for replication” or “conditions effective for amplification” or like phrases used herein contemplates those conditions necessary for catalysis, e.g. those conditions appropriate to permit the catalytic polymerization of nucleic acids taught by the invention. The specific chemical and physical conditions appropriate, suitable or effective for catalysis as practiced in the invention are known or ascertainable by those of skill in the art of nucleic acid detection and assay. Conditions suitable for catalysis include a range of conditions adequate for forming any hybridized nucleic acid species required for replication by the methods of the invention, and catalytically obtaining the replication. The efficiency of the catalysis effected by these conditions is contemplated to be adequate rather than ideal or at a high level. Thus the range of adequate conditions includes both ideal conditions effecting high efficiency catalysis and less than ideal conditions yielding hybridization efficiencies which practicably permit practicing the invention.

The phrase “conditions suitable for hybridization” as used herein contemplates those conditions necessary for hybridization of nucleic acid sequences, e.g. those conditions appropriate to permit double stranded nucleic acid sequences to form from complementary single stranded sequences. The specific chemical and physical conditions appropriate or suitable for hybridization are known or ascertainable by those of skill in the art of nucleic acid detection and assay. Conditions suitable for hybridization include a range of conditions adequate for forming any hybridized nucleic acid species required for replication by the methods of the invention. The stringency of the hybridization effected by these conditions is contemplated to be adequate rather than ideal or at a high level. Thus the range of adequate conditions includes both ideal conditions effecting high stringency hybridization and less than ideal conditions yielding hybridization stringencies which practicably permit detection.

The phrase “DNA-dependent RNA polymerase activity” (DDRP activity) refers to the capability of some RNA polymerases to form RNA from a DNA template. More specifically as pertinent herein, the phrase refers to the ability of some RNA replicases, primarily catalyzing the replication of viral genomic RNA, to use DNA as an alternate template.

The term “amplification probe” as used herein refers to a probe that by itself or as a member of a set of amplification probes for a specific target sequence, (target sequence is defined below) effects the amplification of a specific target sequence. The amplification probes of the invention comprise an anti-target sequence segment having sequence complementary to the target sequence of interest and a replication segment that contains sequence necessary for replication of the target sequence.

The term “detection probe” as used herein refers to a probe used to detect nucleic acid sequence by complementary base pairing hybridization. The detection probes of the invention comprise a sequence complementary to the sequence to be detected and detectable signal or marker indicating the presence of the complementary sequence, for example a chemiluminescent marker, 32P incorporated into the phosphodiester backbone of the nucleic acid sequence or both. Although the method of the invention employs detection probes for target sequence, detection probes for other nucleic acid sequence are also contemplated by detection probe.

A “complex substrate” or “complex hybridized substrate” or “hybridized substrate complex” is a template for replicase mediated replication comprising a hybridized nucleic acid. An example of a typical complex substrate for a RNA replicase is a closed circle of nucleic acid that does not have a free 3′-end, or which has a free 3′-end wherein the sequence segment that is the template for synthesis of a (quasi-)autocatalytically replicase replicable or replicatable RNA catalyzed by the DDRP activity, does not include the 3′-end, or which has a free 3′-end wherein the template for synthesis of a (quasi-)autocatalytically replicable RNA catalyzed by the DDRP activity, includes the 3′-end but has a sequence other than poly-dC at the 3′-end, or which has a free 3′-end wherein the template for (quasi-)auto-catalytic synthesis by the DDRP activity, includes the 3′-end and has a poly-dC at its 3′-end but has sequence other than the poly-dC at the 3′-end, thus comprising at least one 2′-deoxyribonucleotide or analog thereof that is not 2′-dC.

The term “replicase” or “RNA replicase” as employed herein refers to an enzyme that polymerizes RNA to replicate a template requiring the template to form a “complex substrate” as defined above for (quasi-)autocatalytic replication of the complex substrate and template. Replicase enzymes include the Qβ replicase. Although the replicases normally replicate a phage genome that is RNA, they are known to have DNA dependent RNA polymerase (DDRP) activity also, permitting template to be DNA. As used herein replicase contemplates systematically or randomly mutated replicase amino acid sequence, replicases which comprise fusion proteins of a plurality of naturally occurring replicases, and replicases which are otherwise synthetically engineered by combinatorial methods, or based on theoretical bioinformatics, such as thoretical modeling, comparative or homology based prediction or a combination thereof.

The sequence forming the template in a complex substrate for synthesis of an autocatalytically replicatable RNA catalyzed by the DDRP activity of an RNA replicase, is referred to as a “complex segment,” “complex sequence segment” or “complex template.” In the methods of the invention, the “complex segments” preferably comprise at least one 2′-deoxyribonucleotide or analog thereof. The phrase “target sequence” or “target sequence segment” refers to a nucleic acid sequence that is to be detected.

Replicase based procedures are important in amplification of nucleic acids. These methods obtain amplification by the amplification of an amplification probe sequence rather than by direct amplification of a segment or segments of target nucleic acid analyte. Consequently, the amplification probe or probes employed must be carefully designed to both form the correct complex template required by the replicase enzyme, and to effect amplification of the correct probe. One such method is based on the use of the Qβ replicase enzyme and its RNA-dependent RNA polymerase activity. Blumenthal et al. (1979) supra; PCT Patent Publication No. WO 87/06270 and U.S. Pat. No. 4,957,858 to Chu et al.; Feix et al. (1976) supra; Kramer et al. (1989) supra; U.S. Pat. No. 4,786,600 to Kramer; Lizardi, P. M. et al. (1988) supra; and Schaffner et al. (1977), supra generally describe the replicase based amplification procedure. In the procedure, a replicative (referred to as “replicatable”) or replicase replicable RNA molecule is covalently joined to a specific hybridizing probe, a single-stranded nucleic acid having an “anti-target sequence” segment complementary to a “target sequence” segment of the target nucleic acid analyte.

The anti-target sequence segment of the amplification probe may be embedded within a recombinant replicative or replicase replicable (“replicatable”) RNA sequence or attached to one of the ends of a replicative or replicase replicable RNA sequence. The amplification probe, an RNA replicase replicable nucleic acid by virtue of the presence of replicase replicable sequence, hybridizes (by means of the anti-target sequence segment) to target nucleic acid analyte in a sample. The probe-analyte nucleic acid sequence complexes that have hybridized are then typically separated from those that have not. The amplification of probes that hybridize to target to form probe-analyte nucleic acid sequence complexes are amplified exponentially by incubation with Qβ replicase. Qβ replicase catalyzes autocatalytic (more correctly quasi-autocatalytic) replication of the replicase replicable RNA to produce up to 10⁹ reporter molecules (replicatable or replicase replicable RNAs) for each hybridized target molecule. Such amplification can be completed in 30 minutes (Lizardi et al., supra).

The extreme specificity of Qβ replicase for RNAs with certain structural and sequence requirements for catalysis of (quasi-)autocatalytic replication assures that only the replicase replicable RNA associated with probes is amplified (Kramer et al.(1989) supra). Other advantages include the speed of the reaction, the simplicity of manipulations, and that the (quasi-)autocatalytic nature of the process requires no thermal cycling and no initiation steps separate from the general catalytic conditions for replication by a replicase. Qβ replication initiation normally requires a template for which Qβ replicase shows a high degree of template specificity (Werner (1991) Biochemistry 30(24):5832-8; Beibricher (1986) Symposia on Molecular and Cellular Biology New Series v54, Keystone Colo. USA: 9-24; Blumenthal et al. (1980) J. Biol. Chem. 255(24):1713-6; Blumenthal et al. (1979) Annu. Rev. Biochem. 48:525-48). However, a disadvantage arises from the prolific nature of replicase activity, which is known to replicate some unhybridized nucleic acid sequences (Beibricher et al. (1985) Biochemistry 24(23):6550-60) and has even been evidenced to be capable of de novo synthesis of replicase replicable sequence without any template (Beibricher et al. (1987) Cold Spring Harb. Symp. Quant. Biol. 52:299-306; Beibricher et al. (1986) Nature 321(6065):89-91; Hill et al. (1983) Nature 301(5898):350-2; Sumper et al. (1975) Proc. Natl. Acad. Sci. U S A 72(1): 162-6). In addition, except in cases where an anti-target sequence segment can be linked or embedded in a replicative RNA, the target or anti-target sequence segment is not amplified by Qβ replicase.

The present invention preferably employs the DNA dependent RNA polymerase (DDRP) activity of RNA replicases, such as Qβ replicase. Specifically this DDRP activity is with a complex template which comprises a 2′-deoxyribonucleotide or an analog thereof in place of one or more ribonucleotides in a nucleic acid sequence (quasi-)autocatalytically replicable by an RNA replicase. The DDRP activity of the replicase can be utilized to (quasi-)autocatalytically amplify the target/anti-target sequence or a portion thereof. Use of this activity to amplify anti-target/target is practically limited by the ability of the replicase to replicate some unhybridized nucleic acid sequence. For example, an unhybridized amplification probe from a mixture of a number of sets of one or more amplification probes, each set for a specific target, could be amplified. Such unhybridized sequence may result from incomplete separation of hybridized probe-analyte complexes from analyte probe mixtures, or from de novo synthesis of nucleic acids, supra. Together each set of amplification probes comprises the replicase replicable sequence required for the complex, hybridized, template initiation of the RNA replicase.

Higher replicative efficiency and consequently amplification will immediately be appreciated to result from hybridized amplification probes compared to unhybridized amplification probes. This will result in some noise from unhybridized nucleic acid amplification, in addition to signal, representing desired replication hybridizing sequence effected amplification. Often such noise is inconsequential because of the strong signal provided by numerous complex templates formed by hybridization. Such cases do not require additional measures to enhance S/N on order to obtain adequate sensitivity. Using a set of multiple amplification probes that together comprise a complete replicase replicable sequence, and thus must all hybridize to the target sequence to form the complex template, enhances selectivity of amplification directly by making less likely the conjunction of portions of the complete replicase replicable sequence without hybridization. The ability of the replicase to synthesize replicase replicable sequence (and sequence complementary thereto) either de novo or from unhybridized amplification probes ultimately results in significant noise nevertheless. Because Qβ replicase amplification has been shown to occur in the absence of template (e.g. complete replicase replicable sequence), de novo amplification contributes to background signal (noise), despite careful separation of hybridized analyte-amplification probe complexes from the analyte detection assay mixture.

In its simplest embodiment, the invention comprises using detection of amplified anti-target/target rather than of the replicase replicable sequence to enhance the sensitivity of a replicase system employing one or more amplification probes to amplify a complex replicase replicable template. As both target and anti-target sequences are quasi-autocatalytically replicated in the process, either or both may be measured, but preferably target is measured directly to preclude the possibility of ancillary replicative processes that replicate antitarget preferentially. The expectation that such asymmetric replication will not be normally observed engenders the option of measuring both with a mixture of target detection probes, for example emitting the same wavelength and consequently frequency signal.

The method of the instant invention effectively eliminates or filters a large portion of the noise by not measuring as signal any nucleic acid other than a target nucleic acid instead of the customary measurement of the replicase replicable sequence. As de novo synthesis will be predominantly of nucleic acid replicable by the specific RNA replicase, the method of the invention eliminates the majority of noise attributable directly to de novo RNA synthesis. Target sequence is also a small constituent of noise ultimately attributable to unhybridized replication. The predominant non-hybridizing signal or noise not filtered out by the instant invention is ultimately attributable to non-hybridizing replication of the amplification probe comprising the detected target/anti-target sequence. Note that detection of the replicase replicable sequence (by use of detection probe(s) complementary to replicase replicable sequence) has been employed with the invention for comparison to detection of target sequence The spurious signal from non-hybridizing replication of the amplification probe comprising the detected target/anti-target sequence, can be greatly reduced directly by employing multiple amplification probes as is taught in U.S. Pat. No. 6,090,589 to Dimond et al. The kinetic reduction of stable relatively long lived complex template formation is far smaller in magnitude of effect on the signal than the decrement in complete amplification probe sequences comprising both a complete replicase replicable sequence reduces replication of unhybridized sequence. This direct reduction of noise can be combined with the independent filtration effect of detection of the entire sequence enhances S/N multiplicatively, because of the mechanistic independence of the enhancements. Thus combining target/anti-target post amplification detection with the employment of multiple amplification probes together comprising a complete replicase replicable sequence multiplicatively, or synergistically, increases effective amplification and hence sensitivity.

The invention is practiced using a target sequence base pairing specific (therefore comprising complementary anti-target sequence) set of amplification probes. The amplification probe molecules of the set each hybridize to a portion or segment of a specific target sequence of the nucleic acid sample. Each amplification probe of a target specific set of amplification probes comprises an anti-target sequence segment comprising a portion of the anti-target sequence and a replication segment that comprises a portion of a quasi-autocatalytically replicable sequence or a complementary sequence thereto. The replication segments of the target specific set of amplification probes, when taken together, comprise the sequence, or a complementary sequence, of a nucleic acid sequence comprising an RNA replicase quasi-autocatalytically replicable sequence. The complete anti-target/target specific set of amplification probes can hybridize to the sequence of the target molecule. The unhybridized amplification probes are then removed and hybridized amplification probes are subjected to replication conditions resulting in quasi-autocatalytic replication of the entire sequence of the target specific amplification probe set segments and the replicable sequence segments of the corresponding amplification probes. The anti-target sequence segments are then detected using a detection molecule or probe such as a chemiluminescence probe or probes.

The simplest embodiment of the instant invention is practiced as follows. The amplification probe or combination of probes is used to amplify the anti-target and consequently target sequence incorporated in the amplification probe along with a replicase replicable sequence. The amplification probe may additionally comprise additional sequence that may be used, for example, to increase detection specificity. The detection, to increase specificity by filtering noise and consequently enhance effective (or useful considering noise) amplification, is of the specific target/anti-target sequence rather than the replicase replicable sequence, and of any specific sequence added to increase detection specificity. As amplification probes, the method of the invention preferably employs one or more probes to together comprise: (i) an anti-target sequence segment; and (ii) a complete replicase replicable sequence for the replicase enzyme employed.

The invention encompasses numerous practical applications of measuring target rather than replicase replicable sequence, in nucleic acid sequence amplification, target nucleic acid sequence detection, and other fields. In a preferred aspect, the invention employs a method of amplifying a complex nucleic acid segment, which comprises a 2′-deoxyribonucleotide or an analog thereof, and has the nucleic acid sequence which is (quasi-)autocatalytically replicatable by an RNA replicase. Thus employment of more degradation resistant amplification probes comprising DNA is preferred to yield greater amplification and fewer incomplete target/antitarget sequences. The replicase amplification method comprises subjecting a sample which comprises the complex hybridized substrate to conditions effective for (quasi-)autocatalytic replication by the replicase. “hybridized substrate” is defined above.

Conditions effective for (quasi-)autocatalytic replication by an RNA replicase, such as Qβ replicase, are well known or readily ascertained by an ordinarily skilled artisan. Such conditions entail providing in the aqueous solution containing the replicase enzyme, conditions of pH, ionic strength, temperature, and concentration of Mg²+permissive of replicase activity in catalyzing (quasi-)autocatalytic replication and providing as well in the solution the four ribonucleoside 5′-triphosphates (hereinafter referred to simply as “ribonucleoside triphosphates”), which RNA replicases employ as monomer substrates in catalyzing the replication of the complex sequence segment of the complex substrate, the complex. Examples of such conditions are provided in the examples hereinbelow. “Autocatalytic replication” is, as understood in the art, a process catalyzed by an RNA replicase in which a nucleic acid template is employed as a substrate, along with the four ribonucleoside triphosphates, to make an RNA with the sequence complementary to that of the template. Although commonly referred to as autocatalytic the replicase does not replicate itself, thus the process is not autocatalytic in the common sense of the word, but rather quasi-autocatalytic. The term “(quasi-)autocatalytic” and quasi-autocatalytic are therefore used herein interchangeably to denote “autocatalytic” as it is used in the context of replicase amplification art. The RNA molecules made by the replicase also comprise complex template, and can form complex substrate in the replicase amplification process. (Certain ribonucleoside triphosphate analogs, such as TTP or UTP with the 5-carbon of the uracil linked to biotin (see, e.g., Langer et al., Proc. Natl. Acad. Sci. (1981) 78, 6633) may be employed with or in addition to the four standard ribonucleoside triphosphates in autocatalytic replication.) Typically, in a template for DDRP activity of a replicase in accordance with the invention, fewer than 1 in 10 nucleotides will be a 2′-deoxyribonucleotide analog or a ribonucleotide analog. Further, in carrying out DDRP activity on a complex template and quasi-autocatalytic replication of the polynucleotide resulting from the DDRP activity, usually less than about 10 mole % of substrate for the replicase for incorporation into the product of the autocatalytic replication will be analogs of ribonucleoside triphosphates and, more typically, such analogs will be of only one of the four ribonucleoside triphosphates and will be present at less than about 10 mole % of that particular ribonucleoside triphosphate. Preferably, only 2′-deoxyribonucleotides and ribonucleotides will be present in templates for DDRP activity of a replicase and ribonucleoside triphosphates will be used as substrates for DDRP activity and autocatalytic replication.

As indicated above, divalent transition metal ions, such as Mn²⁺, Co²⁺, or Zn²⁺, may also be present to advantage in reaction media in which amplification by a replicase enzyme activity in accordance with the invention is carried out. These ions, as well as the Mg²⁺required for replicase activity, are provided as any salt, which is sufficiently soluble in the solution to achieve the desired metal ion concentration and the anion of which does not reduce replicase enzymatic activity. Suitable salts are well known to the skilled and include the halide salts (e.g., chloride, bromide), the carbonates, the sulfates, the nitrates, and the like.

The invention entails applying the amplification process of the invention in a target-dependent manner, and filtering much of the noise by detecting amplification target sequence rather than the replicase replicable sequence. Thus, practicing the invention entails a method of treating a sample comprising nucleic acid to make a reporter nucleic acid, which is (quasi-)autocatalytically replicatable by an RNA replicase, only if the sample comprises a pre-selected target nucleic acid segment. The method comprises (a) treating a first aliquot of the sample of nucleic acid with one or a set of nucleic acid amplification probes, each capable of hybridizing to a segment of the target sequence or the complement of a segment of the target sequence. If one amplification probe is employed in the method, the amplification probe is, or is capable of being processed to make, a complex nucleic acid substrate comprising a 2′-deoxyribonucleotide or an analog thereof and having the sequence of the amplification probe (reporter) nucleic acid or the complement thereto. If more than one amplification probe is employed in the method, the amplification probes are capable of being processed to make a complex or broken complex nucleic acid sequence comprising a 2′-deoxyribonucleotide or an analog thereof and having the sequence of the reporter RNA or the complement of the reporter RNA. The first aliquot, including the amplification probe or amplification probes is processed to prepare a second aliquot wherein (i) the complex or broken complex nucleic acid sequence comprising a 2′-deoxyribonucleotide or an analog thereof and having the sequence of the reporter RNA or the complement thereof is completed, if not provided as part of a single amplification probe, and remains in an significant amount only if target sequence is present in the sample, and (ii) any nucleic acid sequence, which lacks 2′-deoxyribonucleotides and analogs thereof but is a template for synthesis of reporter nucleic acid or the complement thereof by the RNA replicase, is reduced to an amount that is insignificant. The second aliquot, or a third aliquot taken from the second aliquot, is then subjected to conditions effective for autocatalytic replication in the presence of the replicase. The present invention allows amplification of a target nucleic acid sequence by employing a quasi-autocatalytic replicase activity, while ensuring fidelity of amplification by use of a method for detecting the presence of the amplified target rather than the amplified replicase replicable sequence. An artisan of ordinary skill will appreciate those manipulations such as ligation are preferably employed to complete the complex nucleic acid sequence for promoting maximum stability of the hybridized complex substrate and hence efficiency of (quasi-)autocatalytic replication.

Therefore the invention provides a method for assaying a target nucleic acid comprising combining one or more amplification probes with a nucleic acid sample under conditions suitable for hybridization such that the amplification probe or probes together if more than one probe is used, hybridize to the target sequence. If more than one amplification probe is employed, each probe comprises an anti-target sequence segment, such that each amplification probe hybridizes to a portion of the target sequence of interest that is being probed for in the nucleic acid sample, with each amplification probe comprising an anti-target sequence segment and a replicase replicable sequence segment. When more than one amplification probe is employed, the replicase replicable sequence segments of the amplification probes taken together, have the sequence of, or a complementary sequence to, an RNA sequence which is quasi-autocatalytically replicable by an RNA replicase, resulting in quasi-autocatalytic replication of the entire sequence of the first and second target sequence segments along with the replicase replicable sequence segments of the amplification probes. The anti-target segments are then detected using one or more detection probe molecules, preferably a detection probe comprises a luminescent probe, most preferably a chemiluminescence probe. A set of one or more detection probes for a target sequence may be provided for any target sequence, and thus for both or either strands of the amplified double stranded (DS) target sequence, each set of amplification probes employed together comprising a sequence complementary to one of the strands of the DS target sequence.

Additional detection probes are provided by the invention for determining the amount of unhybridized replicase replicable sequence such that the signal to noise ratio (S/N) between the amplified target segments (signal) and amplified unhybridized probe sequence (noise) can be determined to measure amplification fidelity.

Post-amplification target detection may also be used to “filter” out or decrease measurement of noise and thus enhance the method for assay system in combination with both multiple anti-target/target sequence detection probes. The increased stringency of detection provided by a multiple detection probe system will be readily appreciated to further enhance the noise filtration effect of the instant invention. For maximizing throughput the multiple detection probes should be simultaneously detected. A simultaneous detection method may employ luminescent detection probes and multiple PMT detection, chemiluminescent detection probes are especially convenient and exemplified below. Those skilled in the art will appreciate that numerous simultaneous detection methods may be employed. Simultaneous detection with two detection probes may be conveniently effected by employing a short wavelength emitting chemiluminescent molecule and a long wavelength emitting chemiluminescent molecule.

Although increased numbers of detection probes increase detection stringency, simultaneous detection becomes more difficult, as will be readily appreciated. The ability to increase effective sensitivity and amplification by employing a set of more than one amplification probes, the set of amplification probes together comprising a complete replicase replicable sequence to directly reduce noise, in combination with the independent noise reduction effected by the instant invention will be readily appreciated by those of skill in the art.

Post amplification target detection employing two detection probes combined with a set of two amplification probes for a given target sequence, and detection of anti-target/target sequence by simultaneous hybridization of detection probes with amplified assay products, and simultaneous detection, offers significant gain in effective sensitivity and amplification without complicating the assay or preparation of amplification and detection probes therefor.

Post-amplification target detection may be used to filter out noise and thus enhance the method for assaying a target nucleic acid comprising combining a set of amplification probes, a first and second probe, with a nucleic acid sample under conditions suitable for hybridization such that the first and second probe molecules each hybridize to a portion of a target segment of the nucleic acid sample. Each of the first and second probes comprises an anti-target segment and an amplifiable segment, and the entire sequence of the amplification probes together comprises the complex template sequence segment of the complex hybridized substrate required for (quasi-)autocatalytic replication by an RNA replicase. The anti-target segment of the first and second probe contains a specific sequence that is complementary to and responsible for hybridizing to a portion of a target segment of the nucleic acid sample. The amplifiable segments of the first and second probes, when taken together, have the sequence, or a complementary sequence, which is quasi-autocatalytically replicable by an RNA replicase such as the Qβ replicase. After both the first and second probes have hybridized to the target molecule, the unhybridized probe molecules are preferably removed, and the hybridized probes are subject to the conditions effective for replication by the replicase, resulting in quasi-autocatalytic replication of the entire sequence of the anti-target sequence segments along with the amplifiable sequence segments of the first and second probes. The amplified anti-target segments are then detected using a detection probe molecule, preferably, a luminescent probe, most preferably a chemiluminescent probe. A second detection probe provided by the invention is employed to determine the amount of the amplified amplifiable sequence segments such that the signal to noise ratio (S/N) for the amplified target segments and the signal to noise ratio (S/N) for the amplified amplifiable sequence segments can be determined to measure the fidelity of the amplification.

Post-amplification target detection may also be used to filter out noise and thus enhance the method for assaying a target nucleic acid employing one or a mixture of single amplification probe, each amplification probe comprising a complete replicase replicable sequence, to assay a nucleic acid sample under conditions suitable for hybridization such that the probe molecule hybridizes to a target sequence of the nucleic acid sample. The probe comprises an anti-target sequence segment and a complete replicase replicable segment constructed by routine methods of nucleic acid synthesis. The anti-target sequence portion of the amplification probe contains a specific sequence that is complementary to and consequently hybridizes to a target sequence of the nucleic acid sample. The portion of the amplification probe responsible for its own amplification comprises a complete replicase replicable (replicatable) or “amplifiable” sequence, a sequence, or a complementary sequence thereto, which is (quasi-)autocatalytically replicable by an RNA replicase such as the Qβ replicase.

After the amplification probe has hybridized to the target molecule, unhybridized probe molecules are preferably removed, and the hybridized probe molecules are subject to the conditions effective for replication by the replicase, resulting in quasi-autocatalytic replication of the entire sequence of the anti-target sequence segments along with the amplifiable sequence segments of the probe. The amplified anti-target segment is then detected using a detection probe molecule, preferably, a luminescent probe, most preferably a chemiluminescent probe. A second detection probe provided by the invention is employed to determine the amount of the amplified amplifiable sequence segment such that the signal to noise ratio (S/N) for the amplified target segment and the signal to noise ratio (S/N) for the amplified amplifiable sequence segment can be determined to measure the fidelity of the amplification.

Multiple detection probes for simultaneous hybridization and detection are specifically exemplified below. Simultaneous hybridization of the detected sequence with the multiple detection effects, for a given detection probe in the presence of an additional detection probe, an increase in the slope of the S/N obtained from use of the given probe versus the amount of its corresponding target sequence segment to be detected as compared to the slope of signal to noise ratio obtained for the identical detecting method and protocol except for the absence of the additional detection probe.

Those of skill in the art will immediately appreciate that kits for performing the methods of the invention may be readily assembled. The basic requirements are containers for prepared buffered solutions containing biochemically active replicase, the capture or amplification probe or probes, the detection probes the amplification reaction and the detection assay. The appropriate buffer and reaction solutions for the steps of the invention, including conditions suitable for (quasi-)autocatalytic replication for the amplification of amplification probes, and conditions suitable for hybridization by base pairing complementarity are readily prepared according to the above by artisans of ordinary skill. The replicase enzyme, and amplification and detection probes, and any other reagents can be prepared and stored in appropriately buffered solutions that can be mixed to constitute the desired reaction mixture by routine methods. Conditions include, for example replication and detection conditions such as those permitting nucleic acid hybridization.

It is to be understood that while the invention has been described in conjunction with the preferred specific embodiments thereof, the foregoing description is intended to illustrate and not limit the scope of the invention. Other aspects, advantages and modifications will be apparent to those skilled in the art to which the invention pertains.

All patents, patent applications, journal articles and other references cited herein are incorporated by reference in their entireties.

The following examples are put forth so as to provide those of ordinary skill in the art with a complete disclosure and description of how to implement the invention, and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in °C. and pressure is at or near atmospheric.

### EXAMPLE 1

### Preparation of LEAE Detection Probes

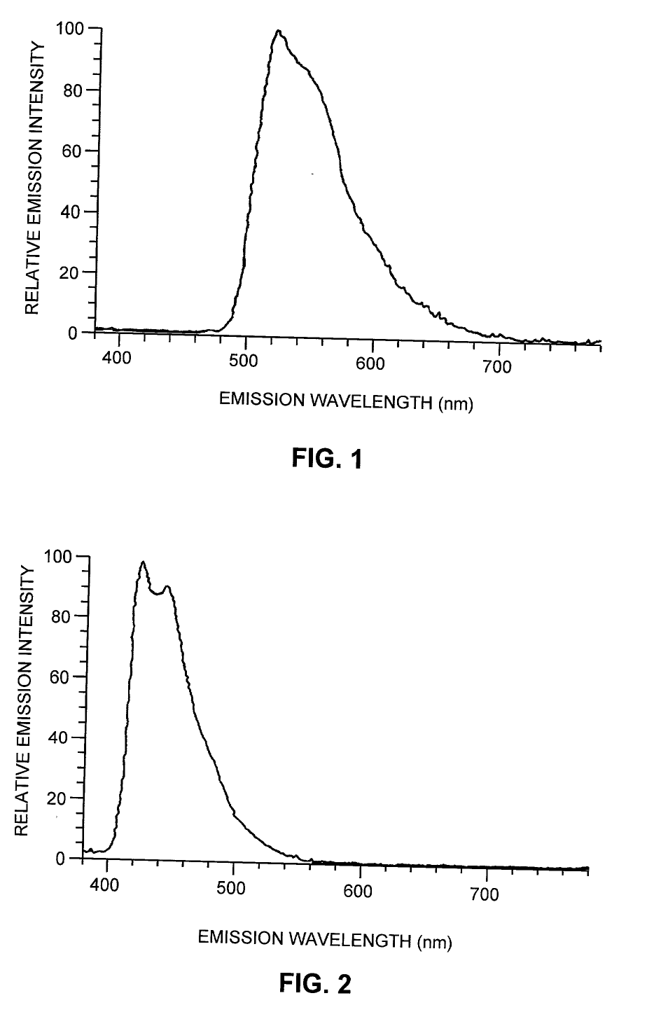

Longer emission acridinium ester N-hydroxy succinamide (LEAE-NHS) and its analogs are disclosed by Law et al. in U.S. Pat. No. 5,395,792. These compounds emit light having an intensity maximum at the wavelength 520 nm (λₘₐₓ=520 nm). FIG. 1 shows the emission spectrum of LEAE-Bz, an analog of LEAE-NHS in this example, which is also disclosed in U.S. Pat. No. 5,395,792. The conjugation of LEAE-NHS to CF10 probe at the 5′ end is described below.

Oligonucleotide CF10 (Sequence: 5′-GT ATC TAT ATT CAT CAT AGG AAA CAC CA) (SEQ ID NO: 1), which has a 5′ amino linker, (20 nmoles) in 0.15 ml of water was treated at room temperature under nitrogen with 0.15 ml of 0.2 M carbonate buffer, pH 8.5 and 0.45 ml of N,N-dimethylformamide (DMF) to give a homogenous solution. To this solution was added a total of 1.9 mg (3.0 nmoles) of LEAE-NHS in 0. 15 ml of DMF in three equal portions, each in a one hour interval. After the addition of the final portion of the LEAE-NHS, the solution was protected from light and stirred at room temperature overnight. The solution was then treated with 2 ml of water and centrifuged at 13,000 RPM for 5 minutes.

The supernatant was passed through a Sepahadex G-25 column (1×40 cm), eluted with water. The very first peak was collected and concentrated in a rotary evaporator at temperature below 35° C. The concentrate was separated on a reverse-phase HPLC column (Brownlee, C-8, RP-300, 4.6×250 mm), eluted with solvent gradient: 5 to 25% B for 15 minutes, followed by 25 to 35% B for 15 minutes, 35 to 60% B for 10 minutes and 60 to 100% B for 5 minutes (A:0.1 M Et₃NHOAc, pH 7.26; B: acetonitrile). The peak with the retention time of ˜34.6 minutes was collected and lyophilized to dryness to give ˜1.43 nmoles of 3′-LEAE-CF10probe as determined from its UV absorbance at 260 nm. The probe was stored in 0.8 ml of 50 mM phosphate buffer, pH 6.0 containing 0.1% Bovine Serum Albumin (BSA) at −20° C. before use.

Oligonucleotide CF1629.27 (Sequence: 5′ AAG ATG ATA TTT TCT TTA ATG GTG CCA) (SEQ ID NO:2) and oligonucleotide 508CF (Sequence: 5′ ATG ATA TTT TCT TTA ATG GTG CCA) (SEQ ID NO:3), both having an amino linker at the 3′ end, were labeled with LEAE at the 3′ end in the manner described above.

### EXAMPLE 2

### Preparation of DMAE-MDV (5′-DMAE-MB65.56) Probe

Dimethyl acridinium esters (DMAE) are disclosed by Law et al. in U.S. Pat. No. 4,745,181. These compounds emit light having an intensity maximum at the wavelength of 430 nm (λₘₐₓ=430 nm). FIG. 2 shows the emission spectrum of DMAE-Bz, which is an analog of DMAE-CO₂H, the compound used in the synthesis of 5′-DMAE-B65.56 described below.

The oligonucleotide, MB65.56 (Sequence: CA CGG GCT AGC GCT TTC GCG CTC TCC CAG GTG ACG CCT CGT GAA GAG GCG CGA CCT (SEQ ID NO: 4) (8.5 nmoles), was treated with triethylamine (536 μmoles) for three hours at room temperature.

The DMAE-CO₂H was activated via mixed anhydride methods disclosed by Law et al. in U.S. Pat. No. 5,622,825, as follows.

DMAE-CO₂H (2.5 mg, 5.36 μmoles) was dissolved in 1.5 ml of DMF and chilled in ice for several minutes. Triethylamine (6 μl, 42.9 μmoles) was added, followed by ethyl chloroformate (2.56 μl, 26.8 μmoles) and stirred, chilled, for half an hour. The reaction mixture was then dried with a rotary evaporator.

The residue was dissolved in DMF and the resulting activated DMAE-CO₂H (850 nmoles) added to the oligonucleotide, in a total volume of 300 μl of 1:1 DMF:H₂O. It was stirred at room temperature overnight.

The reaction mixture was passed through Sephadex G25 (fine) and eluted with water. The first peak was collected, concentrated by rotary evaporation and further purified by HPLC: (Column: Aquapore C8, RP-300, 4.6 mm ×25 cm (Rainin, Woburn, Mass.); Solvents: solvent A: 0.1 M Et₃NHOAc pH 7.2-7.4, solvent B: Acetonitrile; Gradient: (Linear) 8% to 20% B over 20 minutes, to 60% B over 20 minutes; Flowrate: 1 ml/minute; Detection λ:254 nm). A product peak at 27 minutes was collected and lyophilized to give 329 pmoles of the conjugate. The product was stored in 800 μl of 50 mM PO₄, pH 6.0, 0.1% BSA, at −20°C. prior to use.

### EXAMPLE 3

### Preparation of Solid Phase Capture Probe

The solid phase capture probe, PMP-MA, was prepared by immobilizing to paramagnetic particles (PMP) an oligonucleotide capture probe having a sequence 5′GGG GAC CCC CCG GAA GGG GGG ACG AGG TGC GGG CAC CTC GTA CGG GAG TTC GAC CGT GAC A (SEQ ID NO: 5) that is complementary to Midivariant target region A (MA) of the replicase repplicable (replicatable) sequence.

### EXAMPLE 4

### Preparation of the Master Mix

PMP-MA (20 μg/test) were aliquoted to a Sarstead tube, followed by addition of 500 μl of Solid Phase Buffer: 500 mM 2-[N-morpholino]ethanesulfonic acid (MES), 0.1% lithium dodecyl sulphate (LDS), 0.1% 3-[(3-cholamidopropyl)dimethylammonio]-1-propanesulfonate (CHAPS), 0.02% sodium azide (NaN3), 2.4 M lithium chloride (LiCI), 4 ptM tRNA, pH 5.0). The tRNA , commercially obtained, was added to prevent non-specific adsorption or binding of analyte nucleic acid sequence or detection probe(s) by pre-binding or pre-adsorption; that added tRNA should not have any sequence in common with target or anti-target, or any other nucleic acid sequence comprising the detection probe, depending upon the specific detection probe used. The mixture was vortexed, and separated on a Corning Magnetic Separation Unit for 3 minutes. The supernatant was removed by aspiration with a glass Pasteur pipette. The PMP-MA was diluted with the appropriate volume of Solid Phase Buffer to equal 25 μl of buffer/test. To this mixture, the detection probe(s) (200 fmoles/test) were added along with the appropriate volume of Detection Probe Buffer (500 mM MES, 0.1% LDS, 0.1% CHAPS, 0.02% NaN₃, pH 5) to equal 25 μl of probe(s) plus buffer/test. This mixture constituted the Master Mix.

The detection probes were actually added after the aliquoting of the test samples was complete so as to avoid unnecessary time of exposure of the DMAE-or LEAE-conjugated oligonucleotide probes to the solid phase, which may in turn result in increased NSB.

### EXAMPLE 5

### Aliquoting of Samples to Reaction Tubes

Sample Buffer [100 mM tris(hydroxyl)aminomethane (Tris) pH 7.5, 15 mM magnesium chloride (MgCl₂), 0.02% NaN₃], EDTA Sample Buffer, [83 mM Tris pH 7.5, 12.5 mM MgCl₂, 83 mM ethylenediaminetetraacetic acid (EDTA), 0.017% NaN₃) and sample (Qβ amplification product) were sequentially added to a Sarstead tube to result in a final sample volume of 50 μl. EDTA Sample Buffer was used to equalize all the initial sample volumes. A test was run without adding sample and EDTA Sample Buffer only, using the initial sample volume used in the experimental samples, to determine the “noise” value of the assay. This value was used to calculate all of the S/N values in the detection analyses. Sample Buffer was used to bring the final sample volume up to 50 μl. Master Mix (50 μl ) was added to each test. Samples were vortexed on the Ciba Coming Model 4010 Multi-Tube Vortexer. (All vortexing herein was performed on the Model 4010.)

### EXAMPLE 6

### Hybridization Incubation

Incubation was conducted at 60° C. in an MS LAUDA heater (Model M20) for 1 hour. Samples were vortexed in the two 20 minute periods during the incubation. Samples were put on ice immediately following the hybridization incubation period (for about 5-10 minutes).

### EXAMPLE 7

### Separation and Washing of the PMP-MA

Samples were diluted with 400 μl of Wash Buffer (50 mM Tris, 7.5 mM MgCl₂, 250 mM MES, 0.05% LDS, 0.05% CHAPS, 0.6 M LiCl, 0.02% NaN₃). PMP-MA was separated from the supernatant as follows. Samples were vortexed and separated on the Corning Magnetic Separation Unit (hereinafter “Mag Separator”) for 3 minutes. Supernatant was decanted from the tubes using the Mag Separator. Wash Buffer (500 μl) was added to the samples and two wash steps were conducted, as follows.

Samples were vortexed and separated on the Mag Separator for 3 minutes. Supernatant was decanted from the tubes using the Mag Separator. Following the second wash, Wash Buffer (250 μl) was added to the dry PMP-MA. This mixture was vortexed, and 125 μl of the resulting solution was pipetted to a second Sarstead tube.

### EXAMPLE 8

### Light Detection on a Dual PMT Luminometer

The tube containing the above solution was analyzed for LEAE and DMAE chemiluminescent emission signal in a dual photomultiplier tube (PMT) luminometer disclosed in U.S. Pat. No. 5,395,792. The dual PMT luminometer was custom designed to read from both the short and long wavelength probes at the same time. The dual wavelength detection instrument consisted of a chamber where the chemiluminescent reaction occurred and two photomultiplier tubes (PMTs), one on each side of the chamber, so that as the reaction occurred, signals were read in both PMTs simultaneously.

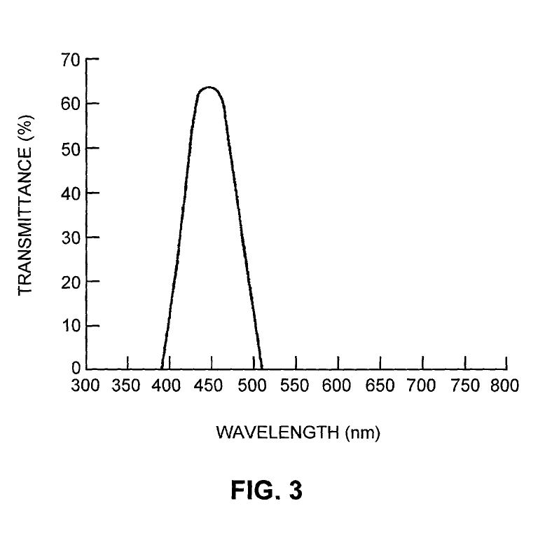

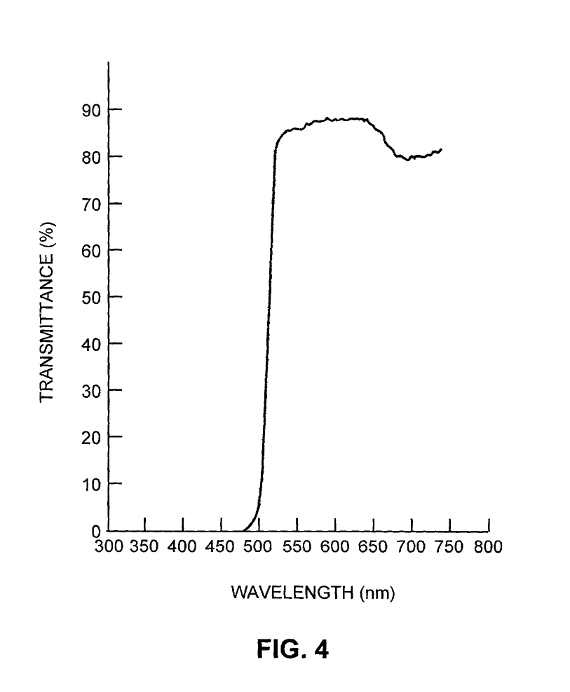

The PMT to detect the DMAE emission signal was fitted with a Corion custom filter (CS-550-F laminated to CS-600-F, Corion Corp., Franklin, MA); the filter's transmittance curve is shown in FIG. 3 as a plot of transmittance versus wavelength. The PMT to detect LEAE was fitted with a Corion 520 filter; the filter's transmittance curve is shown in FIG. 4 as a plot of transmittance versus wavelength. Because a small percentage of signal from DMAE was read in the PMT intended for the LEAE signal and vice versa, data for long and short wavelength signals was corrected in the following way. First, the percentage of either signal in the opposite channel was determined by conducting a reading of the LEAE or DMAE detection probes separately and then data was corrected for “signal cross talk” with the following equations:

Equation I: S(DMAE)=S(s)−k₂*S(LEAE)−b₁; where S(DMAE) is the signal for DMAE, S(LEAE) is the signal for LEAE, S(s) is the total short wavelength signal, k₂ is a constant signifying the proportion of the long wavelength spectrum falling in the range of short wavelength detection and b₁ is a constant signifying instrument background signal in the range of short wavelength detection.

Equation II: S(LEAE)=(S(l)−k1*S(s)+k1*b1−b2)/(1−k₁*k₂); where S(LEAE) is the signal for LEAE, S(l) is the total long wavelength signal, S(s) is the total short wavelength signal, k₁ is a constant signifying the proportion of the short wavelength spectrum falling in the range of short wavelength detection, k₂ is a constant signifying the proportion of the long wavelength spectrum falling tin the range of short wavelength detection, b₁ is a constant signifying instrument background signal in the range of short wavelength detection and b₂ is a constant signifying environmental background signal in the range of long wavelength detection.

### EXAMPLE 9

### Detection of Two Sequences with Dual Probes

In this and all of the examples to follow, Qβ amplification product was generated (as known in the art) using the following two general approaches. (1) MDV (midivariant) (a 221 “mer” or 221 base poly-nucleotide sequence), a natural substrate for the Qβ replicase enzyme was employed as template forming the complex hybridized substrate for (quasi-)autocatalytic replication with Qβ replicase to generate MDV sequence ribonucleotide for use in detection assays to demonstrate the invention in the examples herein. It will be readily appreciated that various nucleic acid sequences sufficiently resembling MDV could have been used in replication. (2) MDV-CF (midivariant with target sequence from the area of the delta F508 mutation on exon 10 of the CFTR (cystic fibrosis transmembrane conductance regulator) gene was generated by Qβ amplification using two amplification probes both comprising Qβ replicase replicable sequence and antitarget sequence complementary to the cystic fibrosis target sequence. These amplification probes were hybridized in the presence of cystic fibrosis target sequence to the target. Then conditions for replication by the replicase were supplied to generate the MDV-CF Qβ replication product used as analyte for demonstration of the invention in the examples.

Typically, amplifications done in the experimental setting herein were conducted with a test analyte obtained by replicase mediated polymerization. An ordinarily skilled artisan will apprehend that many alternatives, including PCR amplification of insert, are available to serve as a target for various replicase amplification experiments described hereinafter. Such test analyte consisted of a nucleic acid nucleotide sequence, typically DNA, which is that of the target double stranded (DS) CFTR sequence or either of the constituent single strands thereof. One of skill will apprehend that the sequence of “antitarget” depends upon which of the DS strands, one or both are to be detected by amplification.

For example both of the strands of the DS sequence could be targeted, in which case two sets of amplification probes must be designed such that amplification probes can not hybridize to each other to form a complex hybridized template. This is not obtainable with sets of amplification probes comprising a single probe because the amplification probe for one strand will hybridize to the amplification probe to form a complete complex hybridized substrate. Two amplification probe sets where the antitarget sequence segments do not overlap may be employed as amplification probes because amplification probes alone can only hybridize to form an incomplete complex substrate that can not be (quasi-)autocatalytically replicated. A small to moderate overlap is also possible when employing two sets of amplification probes, each for one of the DS strands, as long as the overlap is insufficient for both amplification probes from one of the sets to hybridize to one of the amplification probes from the other set to form a complete complex template for hybridization.

In the case of reamplification (i.e. amplification of product from previous amplification), the amplified sequence was the MDV sequence with the target of interest (CF) inserted after position 61. As ribonucleotides are used as the monomer substrate for Qβ amplification of template forming the complex hybridized substrate, the analyte moiety is RNA. Those artisans of ordinary skill in the art will appreciate that either DNA or RNA, and synthetic analogs, including protein nucleic acids, PNAs can be detected as target analyte. For example, RT-PCR (reverse transcriptase PCR) may be employed to pre-amplify a sample of expressed MRNA in cells from tissues obtained directly from an organism or from cultured cells.

5′-DMAE-MB65.56 was used to probe for MDV detection (midivariant replicase replicable sequence segment) and 3′-LEAE-CF1629.27 for target detection with PMP-MA as the solid phase capture probe. Varying amounts of MDV (product of Qβ replicase amplification of full length midivariant sequence) and MDV-CF (product of Qβ replicase amplification using two amplification probes each containing a midivariant replicase replicable sequence segment and target sequence from gene for cystic fibrosis were mixed for each test so that the changing ratio of DMAE/LEAE (or MDV sequence/target sequence) could be observed. MDV was aliquoted in increments of 5 β l (0.7 pmoles) from 0-25 μl while MDV-CF was aliquoted in increments of 5 μl (0.8 pmoles) from 25 to 0 μl in the same tests. Three replicate test series were run, one with both DMAE and LEAE probes, one with DMAE probe only and one with LEAE probe only.

| | Amount of MDV-CF | Amount of MDV |
|---|---|---|
| sample 1 | 4.1 pmoles | — |
| sample 2 | 3.3 pmoles | 0.7 pmoles |
| sample 3 | 2.4 pmoles | 1.4 pmoles |
| sample 4 | 1.6 pmoles | 2.0 pmoles |
| sample 5 | 0.8 pmoles | 2.7 pmoles |
| sample 6 | — | 3.4 pmoles |

Amplifications were conducted with the inclusion of ³²P-CTP to permit quantification of amplification product.

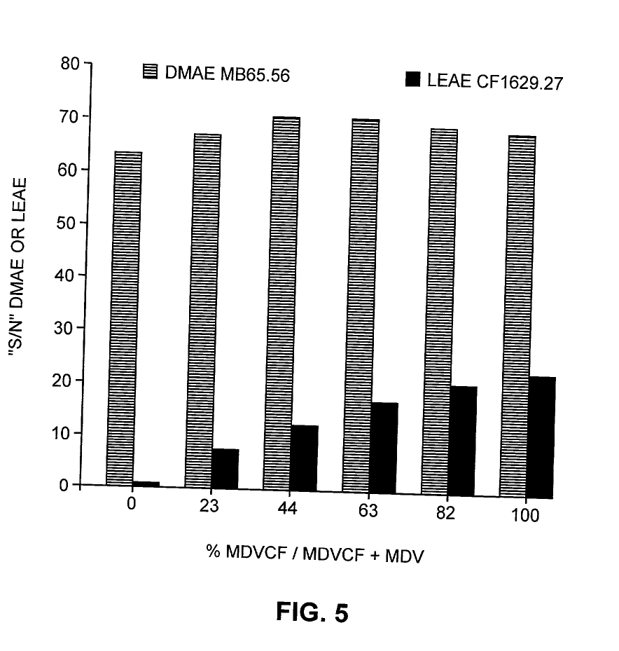

Results are found in TABLE 1, and are also depicted graphically in FIG. 5. For the above six tests, the decreasing S/N for 3′-LEAE-CF1629.27 was observed to be linear (correlation coefficient=0.99). Because both MDV and MDV-CF RNA contain the MDV sequence, the S/N for 5′-DMAE-MB65.56 stayed relatively constant. The S/N value for 3′-LEAE-CF1629.27 for sample 6 (MDV only) was about 1 in test series 1 and 3, and this would be expected since MDV does not contain the CF target sequence. The trend in the S/N values for each of the detection probes with decreasing amounts of MDV-CF and roughly constant amounts of total MDV (MDV-CF plus MDV) in the tests indicates that the “dual label detection” method does specifically measure each sequence in the MDV-CF molecule and data for each sequence is clearly identified by the light signals of different wavelengths emitted by the two chemiluminescent species. It was also observed that in the tests with 3′-LEAE-CF1629.27 probe only, the S/N was lower than the comparative S/N in the analogous tests with both probes.

TABLE 1

| DETECTION PROBE(S) | DMAE S/N | |
|---|---|---|
| EMPLOYED/sample # | (^{S/N}DMAE) | LEAE S/N |
| 5′-DMAE-MB65.56 | | |
| 3′-LEAE-CF 1629.7 | | |
| sample 1 | 68.4 | 24.0 |
| sample 2 | 69.4 | 20.9 |
| sample 3 | 71.0 | 16.3 |
| sample 4 | 70.7 | 12.4 |
| sample 5 | 66.9 | 6.8 |
| sample 6 | 63.3 | 0.9 |
| 5′-DMAE-MB65.56 | | |
| sample 1 | 63.4 | 0.9 |
| sample 2 | 66.6 | 1.1 |
| sample 3 | 69.1 | 1.1 |
| sample 4 | 68.8 | 1.4 |
| sample 5 | 69.2 | 1.3 |
| sample 6 | 72.2 | 1.3 |
| 3′-LEAE-CF 1629.27 | | |
| sample 1 | 0.5 | 16.9 |
| sample 2 | 0.5 | 13.0 |
| sample 3 | 0.6 | 10.7 |
| sample 4 | 0.7 | 7.4 |
| sample 5 | 1.8 | 4.7 |
| sample 6 | 0.9 | 0.8 |

A similar trend in response to the LEAE and DMAE detection probes was observed in the following experiment, which was set up like the experiment above, but with a different target detection probe. In this experiment, 5′-DMAE-MB65.56 was used for the MDV detection probe and 3′-LEAE-508CF for target detection probe with PMP-MA as the solid phase capture probe. Results are shown in TABLE 2.

MDV and MDV-CF were aliquoted for samples 1-5 as below:

| | Amount of MDV-CF | Amount of MDV |
|---|---|---|
| sample 1 | 2.4 pmoles | — |
| sample 2 | 1.6 pmoles | 0.7 pmoles |
| sample 3 | 1.3 pmoles | 1.0 pmoles |
| sample 4 | 0.8 pmoles | 1.4 pmoles |
| sample 5 | — | 2.0 pmoles |

TABLE 2

| DETECTION PROBE(S) | | |
|---|---|---|
| USED IN THE TEST | ^{S/N}DMAE | ^{S/N}LEAE |
| 5′-DMAE-MB65.56 | | |
| 3′-LEAE-508CF | | |
| sample 1 | 31.4 | 5.2 |
| sample 2 | 35.2 | 4.4 |
| sample 3 | 35.3 | 3.8 |
| sample 4 | 34.7 | 2.7 |
| sample 5 | 33.4 | 1.0 |
| 5′-DMAE-MB65.56 | | |
| sample 1 | 33.3 | 1.0 |
| sample 2 | 35.3 | 0.9 |
| sample 3 | 34.0 | 1.0 |
| sample 4 | 33.6 | 1.0 |
| sample 5 | **ND | 0.9 |
| 3′-LEAE-508CF | | |
| sample 1 | 0.9 | 3.1 |
| sample 2 | 0.9 | 2.3 |
| sample 3 | 0.9 | 2.1 |
| sample 4 | 1.0 | 1.6 |
| sample 5 | 1.7 | 0.9 |

**ND = no data

### EXAMPLE 10

### Reproducibility of Results

The dual wavelength detection assay was conducted with 5′-DMAE-MB65.56 as the MDV (replicable sequence) detection probe and with 5′-LEAE-CF 10 as the target detection probe. This target detection probe (which directly abuts the MA sequence in the MDV model) has increased hybridization efficiency over detection probes neighboring the B region of MDV (MB) sequence. In order to examine reproducibility of performance of the dual assay with the 5′-LEAE-CF10 probe, 3 identical experiments were conducted on 3 different days. Six tests were included in each experiment with the amount of MDV-CF decreasing from test 1 (2.7 pmoles) to test 6 (0 pmoles) and the amount of MDV increasing from test 1 (0 pmoles) to test 6 (2.7 pmoles). For each test, the ratio of ^{S/N}LEAE/^{S/N}DMAE was averaged for the 3 experiments. Results are shown in TABLE 3A. CVs ranged from 2% -9%, where the product containing sequences for both of the detection probes was present. Slopes for S/N of LEAE (^{S/N}LEAE) versus amount of MDV-CF (Δ^{S/N}LEAE/ΔMDV-CF) and slope of ^{S/N}DMAE versus total amount of MDV (Δ^{S/N}DMAE/AMDV) were determined for the 3 experiments (TABLE 3B). Ratios of the slopes were averaged for the 3 experiments. Average ratio of the slopes, (Δ^{S/N}LEAE/ΔMDV-CF)/(Δ^{S/N}DMAE/ΔMDV), was 1.75 (SD=0.06, CV=3.5%). The reproducibility of the ratios of ^{S/N}LEAE/^{S/N}DMAE and also the ratio of slopes of the curves, (Δ^{S/N}LEAE/ΔMDV-CF)/(Δ^{S/N}DMAE/ΔMDV), would indicates that the dual label detection method functions to provide comparative data for MDV versus target sequence. The ratio of ^{S/N}LEAE/^{S/N}DMAE can be used to demonstrate “target retention” (fidelity of target replication) in Qβ amplification.

TABLE 3A

| | (amount in pmoles) | | ^{S/N}LEAE/^{S/N}DMAE | | | (n = 3) | |
|---|---|---|---|---|---|---|---|
| Sample # | MDV-CF | MDV | Day 1 | Day 2 | Day 3 | Mean (SD) | % CV |
| 1 | 2.7 | — | 2.01 | 2.19 | 2.06 | 2.09 (0.09) | 4.56% |
| 2 | 2.0 | 0.7 | 2.09 | 2.27 | 2.18 | 2.18 (0.09) | 4.12% |
| 3 | 1.3 | 1.4 | 2.04 | 2.11 | 2.05 | 2.07 (0.04) | 2.01% |
| 4 | 0.7 | 2.0 | 1.70 | 1.75 | 1.99 | 1.81 (0.15) | 8.46% |
| 5 | 0.3 | 2.3 | ND | 1.19 | 1.35 | 1.27 (0.12) | **9.22% |
| 6 | — | 2.7 | 0.08 | 0.10 | 0.14 | 0.10 (0.03) | 29.52% |

**n = 2, ND = no data

TABLE 3B

| | SLOPES OF CURVES | | |
|---|---|---|---|
| | Δ^{S/N}DMAE/ | Δ^{S/N}LEAE/ | (Δ^{S/N}LEAE/ΔMDV-CF)/ |
| | ΔMDV | ΔMDV-CF | (Δ^{S/N}DMAE/ΔMDV) |
| Day 1 | 19.12 | 32.10 | 1.68 |
| Day 2 | 15.95 | 28.38 | 1.78 |
| Day 3 | 13.84 | 24.73 | 1.79_{—————} |
| | | | 1.75 (average) |
| | | | 0.06 (SD) |
| | | | 3.47% (CV) |

### EXAMPLE 11

### Fidelity of Qp Replicase Amplification

Because reproducibility was observed in the ratio of the S/N values for LEAE probe/DMAE probe in experiments where known varying amounts of MDV with and without target were intentionally mixed, the assay of the present invention can be used to predict fidelity of amplification of the target in Qβ amplification.

With a known amount of Qβ amplification product, detection analysis can be conducted and the ratio of the S/N values for LEAE/DMAE can be used to compare to the corresponding values where the relative amounts of MDV with and without target are known. The ratio of the S/N values for LEAE/DMAE can also be compared for one sample against another and any decrease in the ratio can be taken as an indication of loss of the target in the amplification process.

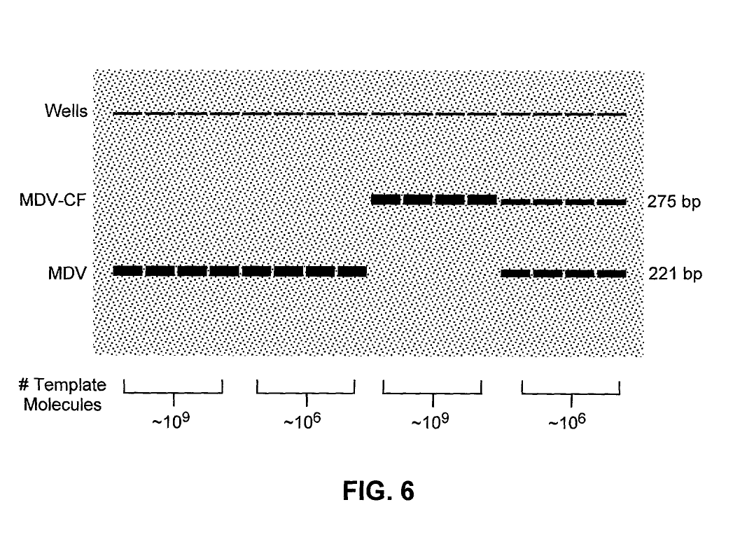

The dual detection assay was used to examine the fidelity of CF target replication in the context of reamplifying Qβ-amplified MDV-CF (product from a first amplification was used as the template in a second amplification). MDV (Qβ amplification product without target) was analyzed the same as was the MDV-CF to serve as control). Two sample types were amplified (four replications per sample type). One sample type had 10⁶ and the second 10⁹ molecules of MDV-CF Qβ replication product (or MDV) and the product was pooled for each starting amount. Five microliters of a 1/1000 dilution of the pooled products were used as the starting template for the second amplification. Both amplifications were conducted with the inclusion of ³²P-CTP to permit quantification of amplification product and corroborative analysis of the chemiluminescense detection data. Following amplification, “dual label detection” analysis was conducted with 5′-DMAE-MB65.56 and 5′-LEAE-CF10 as the MDV and the target detection probes, respectively, and with PMP-MA as the solid phase capture probe. The ratios of the S/N values for LEAE/DMAE, ^{S/N}LEAE/^{S/N}DMAE, were calculated and tabulated in TABLE 4. When 10⁹ molecules were used as starting template amount in the first amplification, the ratios of the ^{SN}LEAE CF10 to ^{S/N}DMAE MB65.56 (^{S/N}LEAE CF10/^{S/N}DMAE MB65.56) using 1.3 pmoles of first amplified MDV-CF and second amplified (reamplified) MDV-CF, were 3.0 and 2.7 respectively. Ratios of the S/N, ^{S/N}LEAE/^{S/N}DMAE using 1.3 pmoles of first and second amplified MDV-CF were 3.3 and 1.1, respectively, when 10⁶ molecules were used for the starting template amount in the first amplification. In the amplification with 10⁶ molecules of starting template, the decrease in ^{S/N}LEAE/^{S/N}DMAE, from the first to the second amplification indicates that the insert target was not retained in 100% of the second amplification (reamplification) product. Using ratios, ^{S/N}LEAE/^{S/N}DMAE from studies with known amounts of MDV-CF/MDV as comparison (TABLE 3A), MDV with the target-containing sequence is estimated to be about 30% of the total MDV from this data. Although this estimation of the percent of MDV containing the target is rough, denaturing gel electrophoresis of Qβ-amplified product corroborated the detection data (FIG. 6).

FIG. 6 shows a denaturing gel electrophoresis of Qβreplicase amplified product. All Qβ amplification product samples run on this gel were from second amplification (reamplification) experiments. Electrophoresis samples in Lanes 1-4 are MDV reamplification product. Product in the second amplification (reamplification) was obtained by Qβ amplification of pooled MDV from the first amplification. The amount of template used for the first amplification was 10⁹ molecules.

Electrophoresis samples in Lanes 5-8 are MDV amplification product (second amplification or reamplification product). Again, the product in the reamplification was obtained by Qβ amplification of pooled MDV from the first amplification, but only 10⁶ molecules were used for the first amplification.

Electrophoresis samples in Lanes 9-12 are MDV-CF reamplification product. Reamplification product was obtained by Qβ amplification of pooled MDV-CF from the first amplification of 10⁹ template molecules.

Electrophoresis samples in Lanes 13-16 are MDV-CF reamplification product. Reamplification product was obtained by Qβ amplification of pooled MDV-CF from the first amplification of 10⁶ molecules.

For MDV-CF reamplification, where 10⁶ molecules of starting template were initially amplified, the presence of a relatively large band at the expected location for MDV (221 mer, without target) was apparent in denaturing gel electrophoresis, which was supported by dual assay detection data. Reamplified MDV (10⁹ and 10⁶ molecules of starting template), was also analyzed in the same electrophoresis gel and the MDV band in these lanes comigrated with one of the bands in lanes with the reamplification product from the MDV-CF sample with 10⁶ molecules of initially amplified starting template. Another band in these lanes comigrates with 275 mer MDV-CF band of lanes 9-12. This two band electrophoretic pattern of lanes 13-16 indicates incomplete retention of the CF insert (target).

Detection data for the reamplification with 10⁹ initially amplified MDV-CF molecules indicated “complete” target retention, and this observation was also supported by results from electrophoresis. Migration of product was comparatively higher than that of the second amplification product discussed above (with 10⁶ molecules of starting template) and migration pattern was at the expected location for MDV-CF (275 mer; with insert as target).

TABLE 4

| | AMOUNT | | |
|---|---|---|---|
| STARTING | OF PRODUCT | RATIO: ^{S/N}LEAE/^{S/N}DMAE | |
| TEMPLATE | IN DETECTION | FIRST | SECOND**** |
| # MOLECULES | ANALYSIS | AMPLIFI- | AMPLIFI- |
| # molecules | (pmoles) | CATION | CATION |
| 1.00 × 10⁹ | 1.3 | 3.0 | 2.7 |
| 1.00 × 10⁹ | 2.7 | 2.6 | |
| 1.00 × 10⁶ | 1.3 | 3.3 | 1.1 |
| 1.00 × 10⁶ | 2.7 | 2.9 | |

****Either 1.00 × 10⁶ or 1.00 × 10⁹ molecules of template were amplified per the standard Qβ amplification protocol (i.e., FIRST AMPLIFICATION). Then, 5 μl of a 1/1000 dilution of the product from the first amplification were used as the template for a second amplification (i.e., SECOND AMPLIFICATION).

### EXAMPLE 12

### Enhancement Of The S/N Of One Detection Probe As A Result Of The Presence Of A Second Probe

It has been observed a number of times with the “dual assay detection” method that the S/N of one detection probe was enhanced as a result of the presence of a second probe.

Using Qβ amplification products (MDV and MDV-CF pooled in varying amounts) as analyte sample, detection analyses were conducted with either one or two detection probes. Enhancement in S/N for one detection probe was demonstrated in those tests where two detection probes were used.

The following three experimental situations were shown to demonstrate the enhancement in response: (i) the enhancement of the SIN of the 3′-LEAE CF1629.27 (3′-LEAE CF1629.27) detection probe as a result of the presence of the 5′-DMAE MB65.56 detection probe; (ii) the enhancement of the S/N of the 3′-LEAE 508CF detection probe as a result of the presence of the 5′-DMAE MB65.56 detection probe (Experiments 2a, 2b, 2c.); (iii) the enhancement of ^{S/N}DMAE MB65.56 detection probe as a result of the presence of the 5′-LEAE CF10 detection probe.

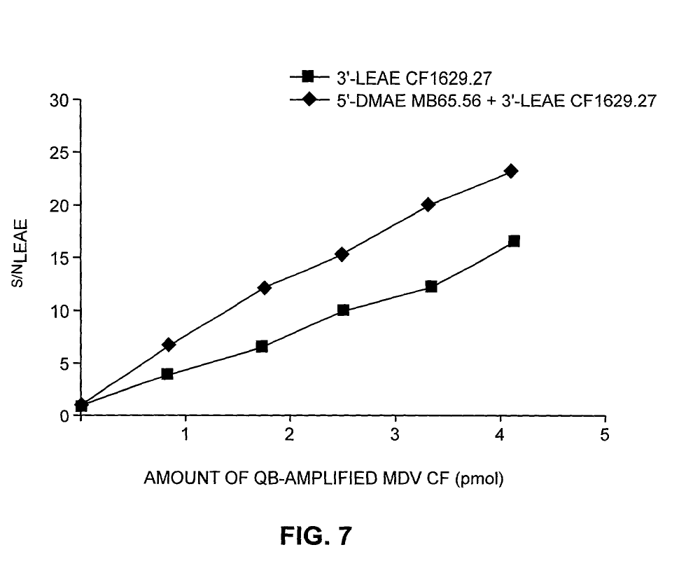

Table 5 contains a summary of the slopes of the S/N of the probe of interest versus the amount of MDV-CF both with and without the presence of the second detection probe. It can be seen that the presence of the second probe yielded from 49% to 105% increase in the slope. FIG. 7 is a graphic demonstration of one such study. Immediately following the summary table, a confirmatory study is described with data from this study presented in Table 6.

Relative location of antitarget in detection assay probes used in obtaining the data in Table 5: CF1629.27 and 508CF are located 2 bases away from MB65.56; 508CF only differs from CF1629.27 by the elimination of three bases from the 5′ side of the antitarget sequence; MB65.56 is 29 bases away from the CF10 probe and CF10 directly abuts the capture probe.

TABLE 5
SUMMARY OF DATA OF ENHANCEMENT IN PERFORMANCE
OF ONE DETECTION PROBE FROM PRESENCE OF A
SECOND DETECTION PROBE

| | SLOPE OF S/N versus amount | | |
|---|---|---|---|
| | of MDV-CF | | |
| | One Detection | Two Detection | |
| Experiment | Probe | Probes | % Enhancement |
| | | | Enhancement of |
| | | | ^{S/N}LEAE |
| | | | CF1629.27 from |
| | | | presence of 5′- |
| | | | DMAE MB65.56 |
| 1 | 3.78 | 5.63 | 49% |
| | | | Enhancement of |
| | | | ^{S/N}LEAE 508CF |
| | | | from presence of |
| | | | 5′-DMAE |
| | | | MB65.56 |
| 2a | 0.92 | 1.80 | 96% |
| 2b | 0.41 | 0.85 | 105% |
| 2c | 0.65 | **0.95 (0.99) | 49% (Average) |
| | | | Enhancement of |
| | | | ^{S/N}DMAE |
| | | | MB65.56 from |
| | | | presence of 5′- |
| | | | LEAE CF10 |
| 3 | 14.18 | 24.47 | 72% |

**Confirmatory study-See below.

The confirmatory study further evidences the observed enhancement of probe performance by the presence of a second probe. By substituting 5′-DMAE-MB65.56 with 5′-OH-MB65.56, it was ascertained that the observation of enhancement is not in any way facilitated by the signal from the DMAE.

The observation of enhancement of the performance of one detection probe by the presence of a second detection probe was confirmed by conducting the “dual detection assay” as described in the following. Three series of tests were performed, each series employing different detection probes (Detection probes: series 1:5′-DMAE-MB65.56 and 3′-LEAE-508CF; series 2:5′-OH-MB65.56 and 3′-LEAE-508CF; and series 3:3′-LEAE-508CF). The data from this study is summarized in TABLE 6.

As is evident, the improvement in S/N values of the 3′-LEAE 508CF detection probe with the presence of a second detection probe (i.e., 5′-DMAE MB 65.56) was unrelated to whether the second probe is conjugated the 5′-DMAE-MB65.56 or unconjugated 5′-OH-MB. This data indicated that the enhancement in S/N resulted from the presence of a second probe and was not somehow facilitated by the presence of a second chemiluminescent signal. In addition, the percent of total RLU bound to the PMP-MA in the detection analysis for the background samples (buffer only) was not substantially different for test series 1, 2, and, 3 (i.e., 1.1%, 1.3%, and 1.3%, respectively). RLU bound to PMP-MA for the background samples was the value used to calculate all of the S/N values in detection analysis. Thus the observed enhancement in S/N may be reasonably concluded to be a result of improved hybridization of the detection probe to the Qβ amplification product.

TABLE 6

| | Amount MDV-CF | Amount MDV |
|---|---|---|
| | (pmoles) | (pmoles) |
| Sample 1 | 2.7 | — |
| Sample 2 | 2.0 | 0.7 |
| Sample 3 | 1.3 | 1.3 |
| Sample 4 | 0.7 | 2.0 |
| Sample 5 | — | 2.7 |
| Employed Detection Probes | ^{S/N}DMAE | ^{S/N}LEAE |
| 5′-DMAE-MB65.56 | | |
| 3′-LEAE-508CF | | |
| sample 1 | 11.0 | 3.7 |
| sample 2 | 8.7 | 2.8 |
| sample 3 | 7.9 | 2.3 |
| sample 4 | 7.0 | 1.8 |
| sample 5 | 5.7 | 1.0 |
| 5′-OH-MB65.56 | | |
| 3′-LEAE-508CF | | |
| sample 1 | 0.7 | 3.3 |
| sample 2 | 0.8 | 3.0 |
| sample 3 | 0.9 | 2.4 |
| sample 4 | 0.9 | 1.6 |
| sample 5 | 1.0 | 0.7 |
| 3′-LEAE-508CF | | |
| sample 1 | 0.9 | 2.6 |
| sample 2 | 0.8 | 2.0 |
| sample 3 | 0.7 | 1.8 |
| sample 4 | 0.7 | 1.2 |
| sample 5 | 0.9 | 0.8 |

We claim:

1. A method of assaying for a target nucleic acid, comprising the steps of:
(a) combining a set of one or more amplification probes with a nucleic acid sample under conditions suitable for hybridization, the nucleic acid sample comprising a target sequence having a complementary antitarget sequence, each amplification probe of the set of amplification probes comprising an antitarget sequence segment, capable of hybridizing to a sequence comprising a portion of the target sequence, and a replication segment comprising partial replicable sequence, such that the set of amplification probes, each by hybridization of the antitarget sequence segment, hybridize to the target sequence to form a hybridized complex, such that set of amplification probes together in the hybridized complex comprise the sequence, or a complementary sequence, of an quasi-autocatalytically replicable sequence;
(b) subjecting the hybridized complex to conditions suitable for activity of the replicase to cause quasi-autocatalytic replication of both the first and second partial replicable sequences and the first and second antitarget sequence segments;
(c) detecting amplified levels of at least one of the replicated antitarget sequence segments.

2. The method of claim 1, wherein the set of one or more amplification probes comprises deoxyribonucleic acid sequences.

3. The method of claim 2, wherein the replicase has a DNA-dependent RNA polymerase activity.

4. The method of claim 3, wherein the replicase is Qβ replicase.

5. The method of claim 3, wherein said detecting of step (c ) is by a set of target sequence detection probes, each target sequence probe comprising a reporter molecule and a detection sequence complementary to at least a portion of the target sequence or the antitarget sequence.

6. The method of claim 5 wherein the detection sequence comprises any portion of the antitarget sequence.

7. The method of claim 5, wherein the reporter molecule comprises a luminescent molecule.

8. The method of claim 7, wherein the luminescent molecule comprises a chemiluminescent molecule.

9. The method of claim 8, wherein the chemiluminescent molecule is selected from the group consisting of acridinium compounds, benzacridinium compounds, quinolinium compounds, isoquinolinium compounds, phenanthridinium compounds, luminol compounds, isoluminol compounds, and lucigenin compounds

10. The method of claim 9 wherein the chemiluminescent molecule is an acridinium compound selected from the group consisting of dimethyl acridinium esters and long emission acridinium esters.

11. The method of claim 1, wherein step (a) further comprises removing all unhybridized first amplification probe and second amplification probe molecules from the hybridized complex.

12. The method of claim 5 further comprising: (d) detecting amplified replicase replicable sequence.

13. The method of claim 12 wherein said detecting of step (d) is by a replicase replicable sequence detection probe comprising a second reporter molecule and a second detection sequence comprising a sequence complementary to any portion of the replicable sequence or the sequence complementary to the replicable sequence.

14. The method of claim 13 wherein the second detection sequence comprises one or a combination of any portion of the first partial replicable sequence and any portion of the second replicable sequence or any sequence complementary thereto.

15. The method of claim 12, wherein the detecting of step (d) is by a replicable sequence detection probe comprising a nucleic acid sequence coupled to a paramagnetic particle, the nucleic acid sequence being complementary to one or a combination of any portion of the first partial replicable sequence and any portion of the second partial replicable sequence or any sequence complementary thereto.

16. The method of claim 13, wherein the second reporter molecule further comprises a luminescent molecule.

17. The method of claim 16 wherein the luminescent molecule comprises a chemiluminescent molecule.

18. The method of claim 17, wherein the chemiluminescent molecule is selected from the group consisting of acridinium compounds, benzacridinium compounds, quinolinium compounds, isoquinolinium compounds, phenanthridinium compounds, luminol compounds, isoluminol compounds, and lucigenin compounds

19. The method of claim 18 wherein the chemiluminescent molecule is an acridinium compound selected from the group consisting of dimethyl acridinium esters and long emission acridinium esters.

20. The method of claim 10, wherein all of the probes comprise deoxyribonucleic acid.

21. A method of assaying for a target nucleic acid, comprising the steps of:
(a) combining a first amplification probe and second amplification probe with a nucleic acid sample under conditions suitable for hybridization, the nucleic acid sample comprising a target sequence having a complementary antitarget sequence, the first amplification probe comprising a first antitarget sequence segment capable of hybridizing to a portion of the target sequence and a first partial replicable sequence, the second probe comprising a second antitarget sequence segment which is capable of hybridizing to a portion of the target segment and a second partial replicable sequence, such that the first and second antitarget sequence segments hybridize to the target sequence to form a hybridized complex, such that the first and second partial replicable sequences together in the hybridized complex have the sequence, or a complementary sequence, of an quasi-autocatalytically replicable sequence;
(b) subjecting the hybridized complex to conditions suitable for activity of the replicase to cause quasi-autocatalytic replication of both the first and second partial replicable sequences and the first and second antitarget sequence segments;
(c) detecting amplified levels of at least one of the replicated first and second antitarget sequence segments.

22. The method of claim 21, wherein the first and second amplification probes comprise deoxyribonucleic acids.

23. The method of claim 22, wherein the replicase has a DNA-dependent RNA polymerase activity.

24. The method of claim 23, wherein the replicase is Qβ replicase.

25. The method of claim 23, wherein said detecting of step (c) is by a target sequence detection probe comprising a first reporter molecule and a first detection sequence complementary to at least a portion of the target sequence or the antitarget sequence.

26. The method of claim 25 wherein the first detection sequence comprises one or a combination of any portion of the first antitarget sequence segment and any portion of the second antitarget sequence segment.

27. The method of claim 25, wherein the first reporter molecule further comprises a luminescent molecule.

28. The method of claim 27, wherein the luminescent molecule comprises a chemiluminescent molecule.

29. The method of claim 28, wherein the chemiluminescent molecule is selected from the group consisting of acridinium compounds, benzacridinium compounds, quinolinium compounds, isoquinolinium compounds, phenanthridinium compounds, luminol compounds, isoluminol compounds, and lucigenin compounds.

30. The method of claim 29 wherein the chemiluminescent molecule is an acridinium compound selected from the group consisting of dimethyl acridinium esters and long emission acridinium esters.

31. The method of claim 21, wherein step (a) further comprises removing all unhybridized first amplification probe and second amplification probe molecules from the hybridized complex.

32. The method of claim 25 further comprising: (d) detecting amplified replicable sequence.

33. The method of claim 32 wherein said detecting of step (d) is by a replicable sequence detection probe comprising a second reporter molecule and a second detection sequence comprising a sequence complementary to any portion of the replicable sequence or the sequence complementary to the replicable sequence.

34. The method of claim 33 wherein the second detection sequence comprises one or a combination of any portion of the first partial replicable sequence and any portion of the second replicable sequence or any sequence complementary thereto.

35. The method of claim 12, wherein the detecting of step (d) is by a replicable sequence detection probe comprising a nucleic acid sequence coupled to a paramagnetic particle, the nucleic acid sequence being complementary to one or a combination of any portion of the first partial replicable sequence and any portion of the second partial replicable sequence or any sequence complementary thereto.

36. The method of claim 33, wherein the second reporter molecule further comprises a luminescent molecule.

37. The method of claim 36 wherein the luminescent molecule comprises a chemiluminescent molecule.

38. The method of claim 37, wherein the chemiluminescent molecule is selected from the group consisting of acridinium compounds, benzacridinium compounds, quinolinium compounds, isoquinolinium compounds, phenanthridinium compounds, luminol compounds, isoluminol compounds, and lucigenin compounds.

39. The method of claim 38 wherein the chemiluminescent molecule is an acridinium compound selected from the group consisting of dimethyl acridinium esters and long emission acridinium esters.

40. The method of claim 30, wherein all of the probes comprise deoxyribonucleic acid.

41. A method of assaying for a target nucleic acid, comprising the steps of:
(a) combining a complete amplification probe with a nucleic acid sample under conditions suitable for hybridization, the nucleic acid sample comprising a target sequence, the complete amplification probe comprising an antitarget sequence which is capable of hybridizing to a portion of the target sequence and a replicable sequence or a complementary sequence thereto, whereby the antitarget sequence hybridizes to the target sequence, such that the replicable sequence comprises a nucleic acid sequence which is quasi-autocatalytically replicable;
(b) subjecting the amplifiable segments to conditions effective for catalysis with the replicase resulting in quasi-autocatalytic replication of both the amplifiable sequence and the antitarget sequence;
(c) detecting the presence of the replicated antitarget sequence.

42. The method of claim 41, wherein the complete amplification probe comprises deoxyribonucleic acid.

43. The method of claim 42, wherein the replicase is a replicase having a DNA-dependent RNA polymerase activity.

44. The method of claim 41, wherein the replicase is Qβ replicase.

45. The method of claim 43 further comprising (d) combining the replicated antitarget sequence with a detection probe comprising a reporter molecule and a detection sequence, the detection sequence having the sequence, or complementary sequence, of a portion of the antitarget sequence.

46. The method of claim 45, wherein the reporter molecule further comprises a luminescent molecule.

47. The method of claim 46, wherein the luminescent molecule is a chemiluminescent molecule.

48. The method of claim 46, wherein the luminescent molecule is a flourescent molecule.

49. The method of claim 47, wherein the chemiluminescent molecule is selected from the group consisting of acridinium compounds, benzacridinium compounds, quinolinium compounds, isoquinolinium compounds, phenanthridinium compounds, luminol compounds, isoluminol compounds, and lucigenin compounds

50. The method of claim 20, wherein the chemiluminescent molecule is an acridinium compound selected from the group consisting of of dimethyl acridinium esters and long emission acridinium esters.

51. The method of claim 41, wherein step (a) further comprises removing all unhybridized complete amplification probe molecules from the nucleic acid sample.

52. The method of claim 41, wherein all of the probes comprise deoxyribonucleic acid.

53. A kit for nucleic acid amplification, comprising:
(a) a set of one or more amplification probe containers, each container containing one amplification probe of a set of target specific amplification probes, each amplification probe comprising an antitarget sequence segment which is capable of hybridizing to a portion of a target nucleic acid sequence and a replicase replicable sequence segment, whereby the antitarget sequence segment of each amplification probe hybridizes to the target sequence, such that the set of amplification probes together comprise a quasi-autocatalytically replicable sequence, or a complementary sequence thereto;
(b) a replicase enzyme container; and
(c) a detection probe container holding one or more detection probes, each detection probe comprising a reporter molecule and a target sequence detection segment, the target sequence detection seghaving the sequence of a portion of one or more of the first antitarget sequence and the second antitarget sequence, or a complementary sequence thereto.

54. The kit of claim 53, wherein the amplification probes comprise deoxyribonucleic acids.

55. The kit of claim 54, wherein the replicase is a replicase having a DNA-dependent RNA polymerase activity.

56. The kit of claim 55, wherein the replicase is Qβ replicase.

57. The kit of claim 55, wherein said detecting of step (c) is by a target sequence detection probe comprising a first reporter molecule and a first detection sequence complementary to at least a portion of the target sequence or the antitarget sequence.

58. The kit of claim 57 wherein the first detection sequence comprises any portion of the antitarget sequence.

59. The kit of claim 57, wherein the first reporter molecule further comprises a luminescent molecule.

60. The kit of claim 59 wherein the luminescent molecule comprises a chemiluminescent molecule.

61. The kit of claim 60, wherein the chemiluminescent molecule is selected from the group consisting of acridinium compounds, benzacridinium compounds, quinolinium compounds, isoquinolinium compounds, phenanthridinium compounds, luminol compounds, isoluminol compounds, and lucigenin compounds.

62. The kit of claim 60 wherein the chemiluminescent molecule is an acridinium compound selected from the group consisting of dimethyl acridinium esters and long emission acridinium esters.

63. The kit of claim 53, wherein step (a) further comprises removing all unhybridized first amplification probe and second amplification probe molecules from the hybridized complex.

64. The kit of claim 57 further comprising: (d) detecting amplified replicable sequence.

65. The kit of claim 64 wherein said detecting of step (d) is by a replicable sequence detection probe comprising a second reporter molecule and a second detection sequence comprising a sequence complementary to any portion of the replicable sequence or the sequence complementary to the replicable sequence.

66. The kit of claim 65 wherein the second detection sequence comprises any portion of the replicase replicable sequence or any sequence complementary thereto.

67. The kit of claim 66, wherein the detecting of step (d) is by a replicase replicable sequence detection probe comprising a nucleic acid sequence coupled to a paramagnetic particle, the nucleic acid sequence being complementary to any portion of the replicase replicable sequence or any sequence complementary thereto.

68. The kit of claim 65, wherein the second reporter molecule further comprises a luminescent molecule.

69. The kit of claim 68 wherein the luminescent molecule comprises a chemiluminescent molecule.

70. The kit of claim 69, wherein the chemiluminescent molecule is selected from the group consisting of acridinium compounds, benzacridinium compounds, quinolinium compounds, isoquinolinium compounds, phenanthridinium compounds, luminol compounds, isoluminol compounds, and lucigenin compounds.

71. The kit of claim 70 wherein the chemiluminescent molecule is an acridinium compound selected from the group consisting of dimethyl acridinium esters and long emission acridinium esters.

72. The kit of claim 67, wherein all of the amplification and detection probes comprise deoxyribonucleic acid.

73. A method of increasing the signal to noise ratio for detecting a target sequence obtainable from a first detection probe that hybridizes to a first target sequence segment comprising employing a second detection probe that hybridizes to a second target sequence segment,
wherein the second target sequence segment does not overlap in sequence with the first target sequence segment, both the first target sequence segment and the second target sequence segment residing in a region comprising thee target sequence and the signal to noise ratio for detection of the target from the first detection probe taken alone is enhanced by the presence of the second detection probe.

74. The method of claim 73 wherein the target sequence is a double stranded sequence.

75. The method of claim 73 further comprising employing a third detection probe that hybridizes to a third target sequence segment,
wherein the third target sequence segment does not overlap in sequence with either the first target sequence segment or the second target sequence segment, and the signal to noise ratio for detection of the target from the first detection probe taken alone is enhanced by presence of the third detection probe in addition to presence of the fsecond detection probe.

76. The method of claim 73, wherein at least one detection probe comprises a deoxyribonucleic acid sequence.

77. The method of claim 73, wherein said detecting is by a set of target sequence detection probes, each target sequence probe comprising a reporter molecule and a detection sequence complementary to at least a portion of the target sequence.

78. The method of claim 77, wherein the detection sequence comprises any portion of an antitarget sequence.

79. The method of claim 78, wherein the reporter molecule further comprises a luminescent molecule.

80. The method of claim 79, wherein the luminescent molecule comprises a chemiluminescent molecule.

81. The method of claim 80, wherein the chemiluminescent molecule is selected from the group consisting of acridinium compounds, benzacridinium compounds, quinolinium compounds, isoquinolinium compounds, phenanthridinium compounds, luminol compounds, isoluminol compounds, and lucigenin compounds.

82. The method of claim 81, wherein the chemiluminescent molecule is an acridinium compound selected from the group consisting of dimethyl acridinium esters and long emission acridinium esters.
